(19) United States
(12) Patent Application Publication
(10) Pub. No.: US 2011/0230663 A1
(43) Pub. Date: Sep. 22, 2011

(54) PROCESS FOR THE PREPARATION OF DONEPEZIL HYDROCHLORIDE

(75) Inventors: Ravi Ponnaiah, Gujarat (IN); Ashok Prasad, Gujarat (IN); Dineshkumar Ramabhai Panchasara, Gujarat (IN); Gajanan Jijaba Chavan, Gujarat (IN); Bhavesh Hareshbhai Prajapati, Gujarat (IN); Ilesh Mahendrabhai Patel, Gujarat (IN)

(73) Assignee: ALEMBIC LIMITED, Gujarat, Vadodara (IN)

(21) Appl. No.: 13/130,810

(22) PCT Filed: Nov. 30, 2009
(86) PCT No.: PCT/IB2009/055414

§ 371 Date: May 24, 2011

(30) Foreign Application Priority Data
Dec. 15, 2008 (IN) 2599/MUM/2008

(51) Int. Cl. C07D 211/06

(52) U.S. Cl. 546/206

(57) ABSTRACT

The present invention relates to novel process for preparing Donepezil hydrochloride of formula (I)

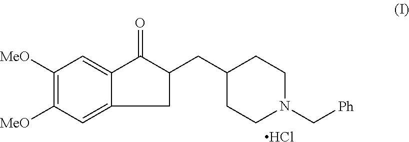

### FIELD OF THE INVENTION

The present invention relates to novel process for preparing Donepezil hydrochloride of formula (I)

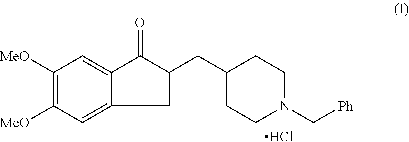

### BACKGROUND OF THE INVENTION

The chemical name of Donepezil is 2,3-Dihydro-5,6-dimethoxy-2-[[1-(phenylmethyl)-4-piperidinyl]methyl]-1H-inden-1-one and formula is C₂₄H₂₉NO₃ and molecular weight is 379.49. The drug is used in its Hydrochloride salt. The current pharmaceutical product containing this drug is being sold by Eisai using the tradename Aricept, in the form of oral tablets and orally disintegrating tablet.

Donepezil is acetylcholinesterase inhibitor class drug. It is used in the treatment of Alzheimer disease. It is also used in the treatment of cognitive defect, attention deficit disorder, migraine and dementia.

U.S. Pat. No. 4,895,841 describes a process for the preparation of Donepezil hydrochloride which is shown in the scheme-I:

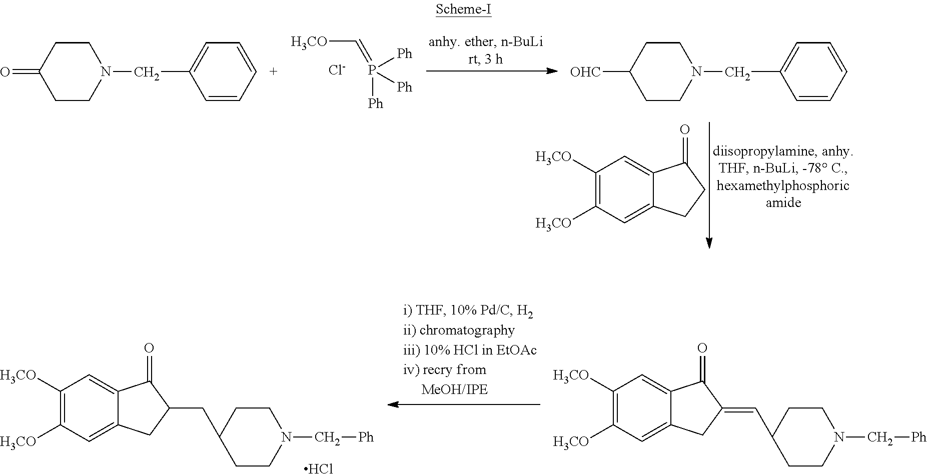

U.S. Pat. No. 5,606,064 describes a process for the preparation of Donepezil hydrochloride which is shown in the scheme-II:

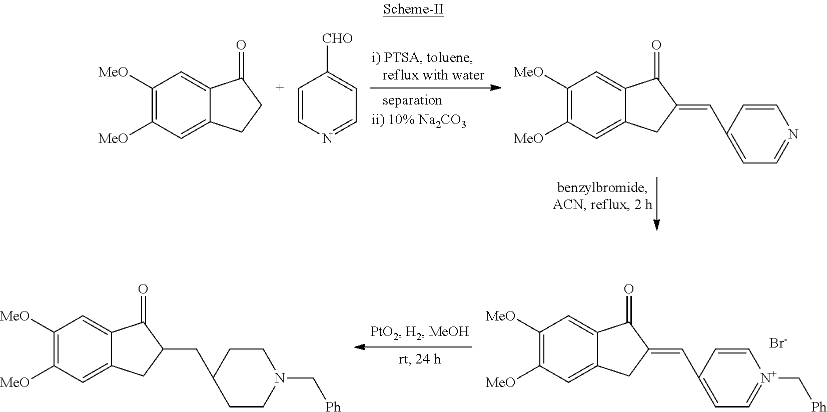

U.S. Pat. No. 7,148,354 describes a process for the preparation of Donepezil hydrochloride which is shown in the scheme-III:

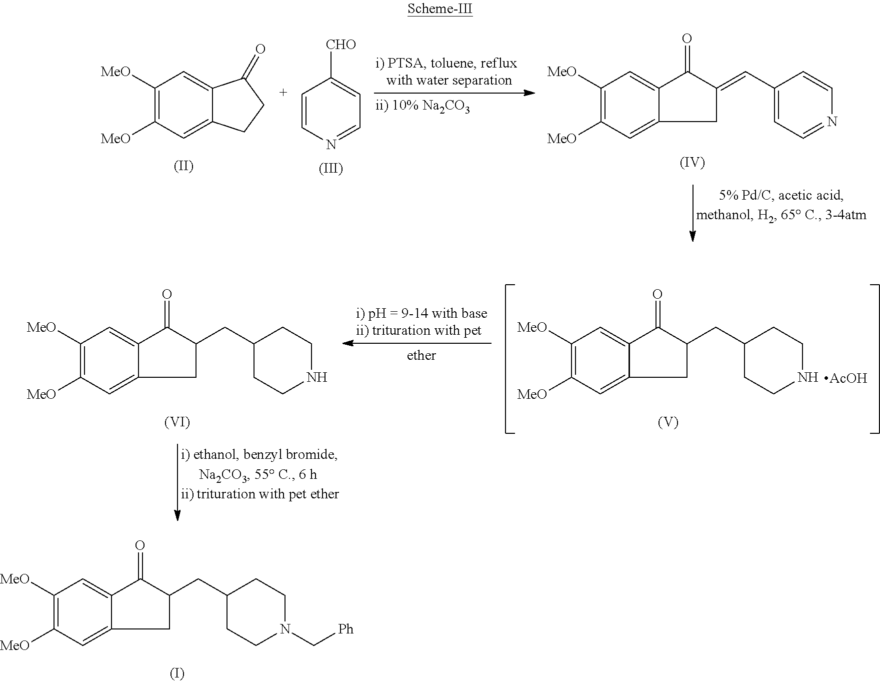

US patent application no. 20070129549 describes a process for the preparation of Donepezil hydrochloride which is shown in the scheme-IV:

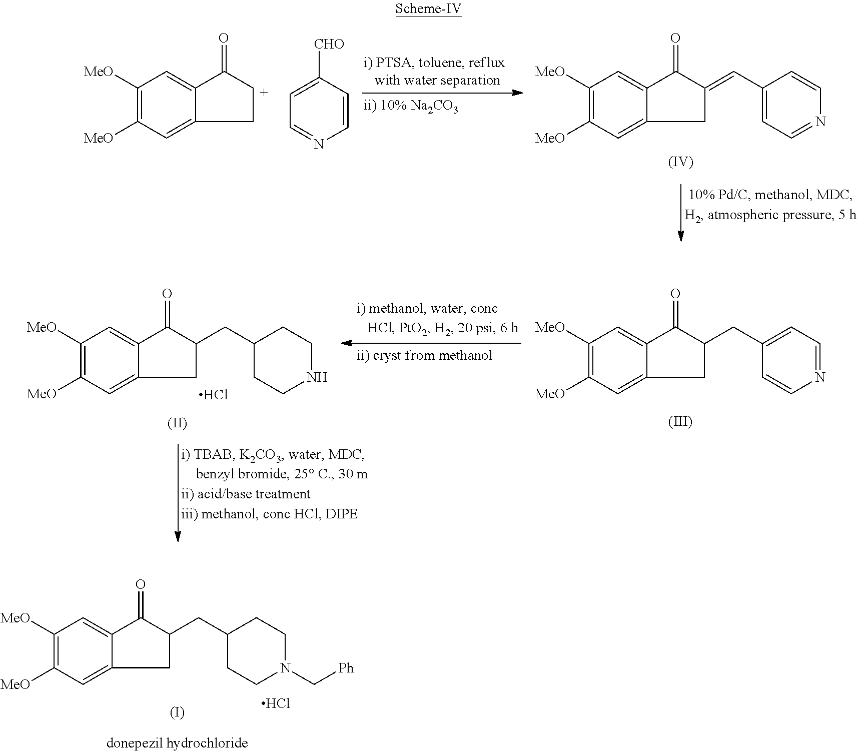

PCT application no 2005076749 describes a process for the preparation of Donepezil which is shown in the scheme-IV:

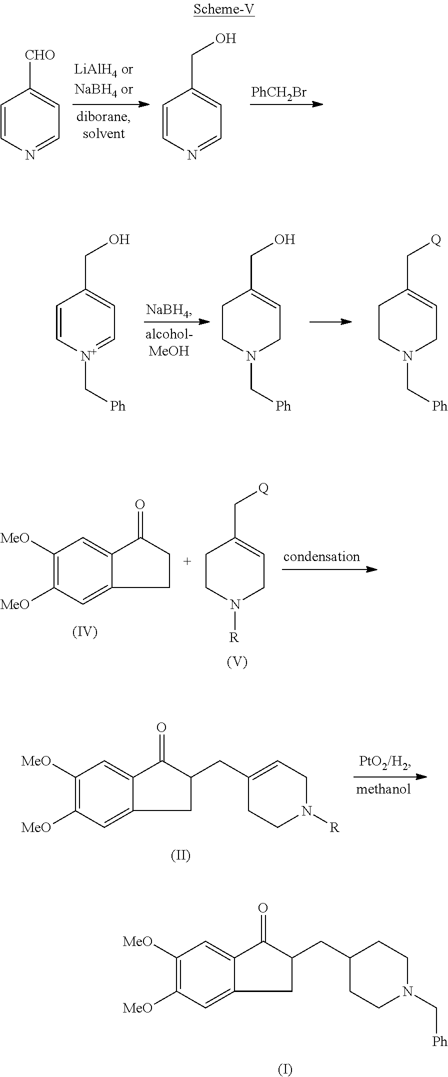

The above processes suffer one or more drawbacks such as use of costly, hazardous reagents or easily flammable reagent which require specialized equipment and due care. Some process reports low yield whereas other reports low purity. The above processes have large number of steps which increases the overall cost of the production. Therefore, above processes are industrially not suitable.

It is therefore, a need to develop an easy to operate, industrially feasible process which also provides high yield and purity of Donepezil hydrochloride. The present invention addresses these needs.

Present inventors have directed their research work towards developing a new process for the preparation of Donepezil hydrochloride using novel intermediate of formula (VII). The process of the present invention provides high yield and purity of Donepezil hydrochloride.

### OBJECT OF THE INVENTION

Accordingly, it is an object of the present invention to provide a novel process for the preparation of Donepezil hydrochloride with high yield and purity.

Another object of the present invention is to provide novel intermediate of formula (VII)

Another object of the present invention is to provide a novel process for the preparation of Donepezil hydrochloride using novel intermediate of formula (VII)

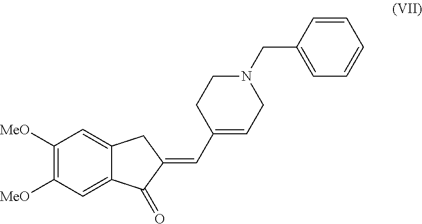

Another object of the present invention is to provide a novel process for the preparation of Donepezil hydrochloride which is operationally simple and cost effective.

### SUMMARY OF THE INVENTION

One aspect of the present invention provides a process for preparation of Donepezil hydrochloride of formula (I)

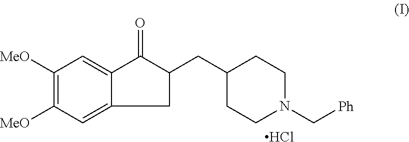

comprising a step of condensing bromide salt of formula (IV) with indanone of formula (V)

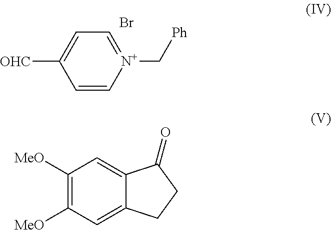

to obtain condensed bromide salt of formula (VI)

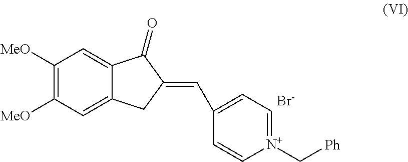

Another aspect of the present invention provides a process for preparation of Donepezil hydrochloride of formula (I)

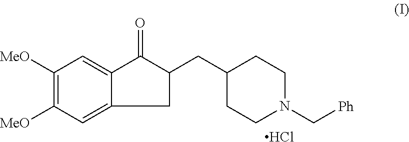

comprising a step of reducing condensed bromide salt of formula (VI) to obtain Di-Ene intermediate of formula (VII)

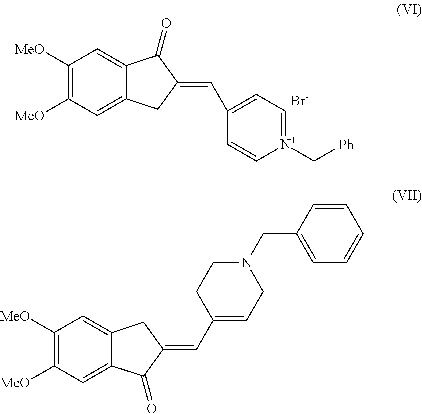

Another aspect of the present invention provides a process for preparation of Donepezil hydrochloride of formula (I)

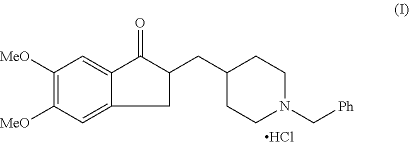

comprising a step of reducing Di-Ene intermediate of formula (VII) to obtain donepezil free base of formula (VIII)

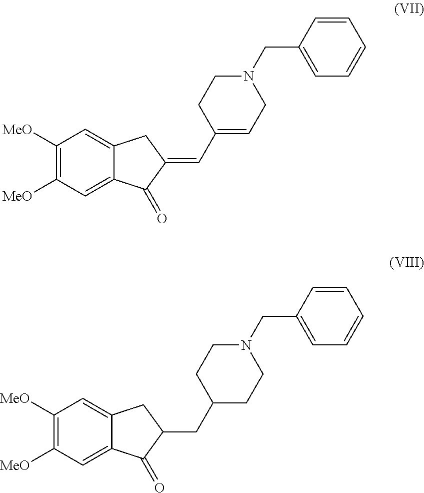

Another aspect of the present invention provides a novel Di-Ene intermediate of formula (VII)

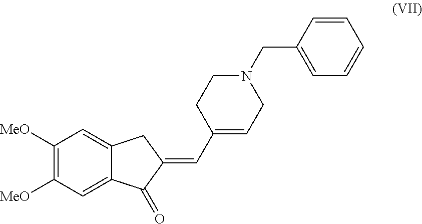

Another aspect of the present invention provides a process for the preparation of novel intermediate of formula (VII) comprising steps of:
(a) condensing pyridine-4-carboxaldehyde of formula (II) with benzyl bromide of formula (III)

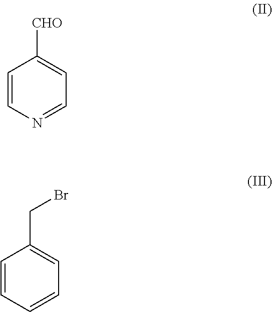

to obtain bromide salt of formula (IV)

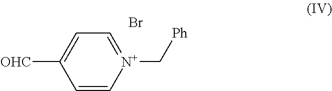

(b) condensing bromide salt of formula (IV) with indanone of formula (V) to obtain condensed bromide salt of formula (VI)

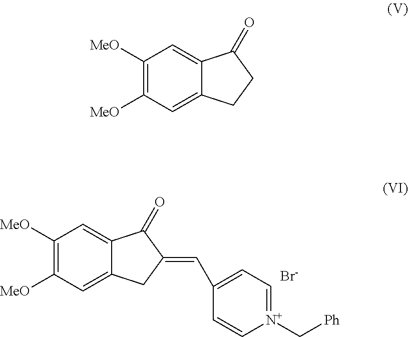

(c) reducing condensed bromide salt of formula (VI) to obtain novel Di-Ene intermediate of formula (VII)

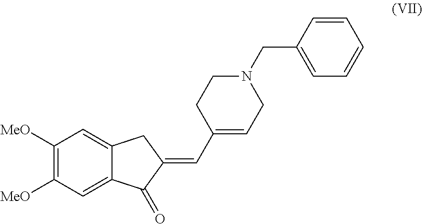

Another aspect of the present invention provides use of novel intermediate of formula (VII) for the preparation of Donepezil hydrochloride of formula (I)

Another aspect of the present invention provides a process for preparation of Donepezil hydrochloride of formula (I)

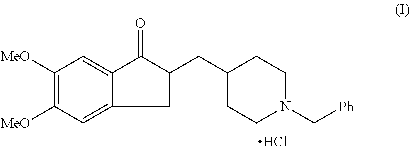

comprising purification of Di-ene intermediate of formula (VII)

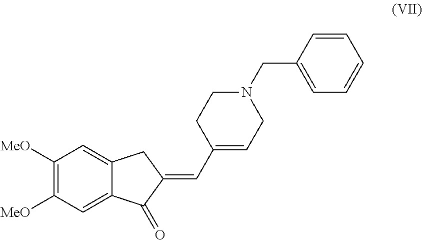

comprising crystallizing crude Di-ene from a mixture of ethyl acetate, acetone, D M Water and ammonia.

Further aspect of the present invention provides a process for preparation of Donepezil hydrochloride of formula (I)

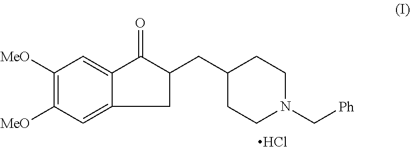

comprising steps of:
(a) condensing pyridine-4-carboxaldehyde of formula (II) with benzyl bromide of formula (III)

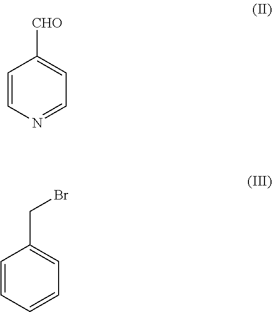

to obtain bromide salt of formula (IV)

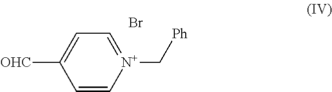

(b) condensing bromide salt of formula (IV) with indanone of formula (V) to obtain condensed bromide salt of formula (VI)

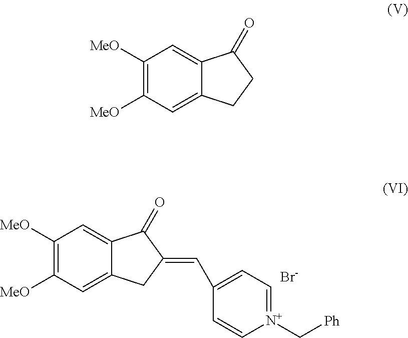

(c) reducing condensed bromide salt of formula (VI) to obtain novel Di-Ene intermediate of formula (VII)

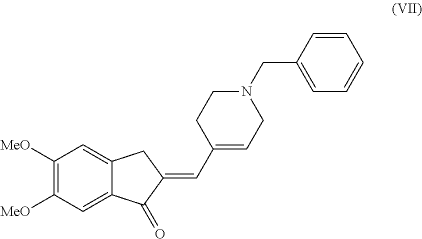

(d) reducing Di-Ene intermediate of formula (VII) to obtain donepezil free base of formula (VIII)

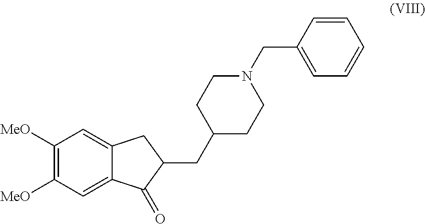

(e) converting donepezil free base to Donepezil hydrochloride of formula (I).

Still further aspect of the present invention provides a process for preparation of Donepezil hydrochloride of formula (I)

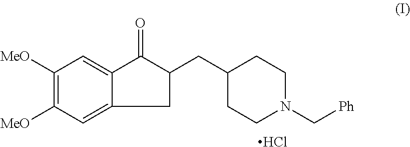

comprising steps of:
(a) condensing pyridine-4-carboxaldehyde of formula (II) with benzyl bromide of formula (III) to obtain bromide salt of formula (IV)
(b) condensing bromide salt of formula (IV) in situ with indanone of formula (v) to obtain condensed bromide salt of formula (VI)

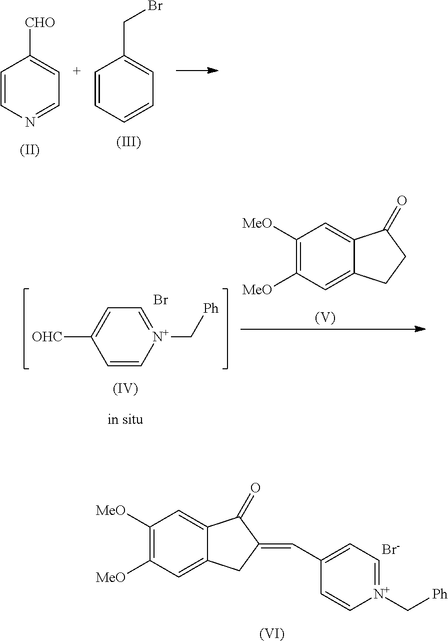

### DETAILED DESCRIPTION OF THE INVENTION

The present invention provides process for preparation of Donepezil hydrochloride of formula (I)

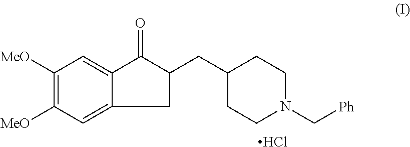

comprising steps of:
(a) condensing pyridine-4-carboxaldehyde of formula (II) with benzyl bromide of formula (III)

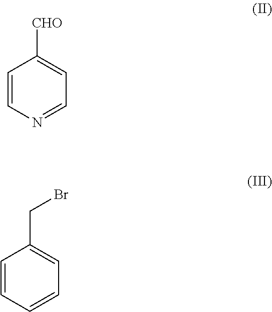

to obtain bromide salt of formula (IV)

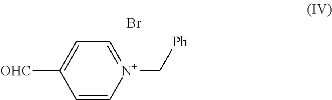

(b) condensing bromide salt of formula (IV) with indanone of formula (V) to obtain condensed bromide salt of formula (VI)

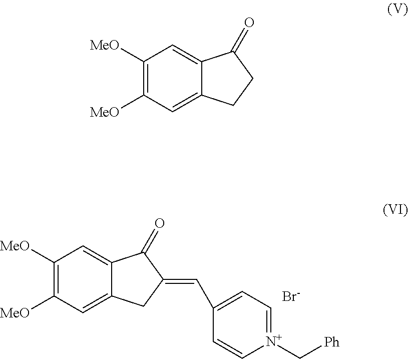

(c) reducing condensed bromide salt of formula (VI) to obtain novel Di-Ene intermediate of formula (VII)

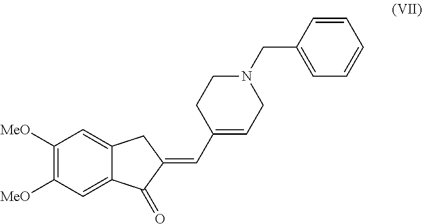

(d) reducing Di-Ene intermediate of formula (VII) to obtain donepezil free base of formula (VIII)

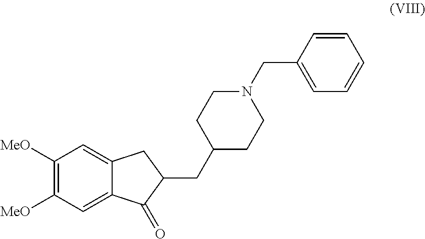

(e) converting donepezil free base to Donepezil hydrochloride of formula (I).

Pyridine-4-carboxaldehyde in N,N-dimethyl formamide (DMF) is cooled to at a temperature of about 10° C. to about 15° C. Benzyl Bromide is added during 30 min. Exotherm is observed in the reaction mixture. The reaction mixture is heated to a temperature of about 60° C. to about 65° C. for 30 to 40 min. The progress of the reaction is monitored by thin layer chromatography (TLC). After completion of reaction on TLC, the reaction mixture is cooled to ambient temperature and used for next step which is optionally carried out in situ. Acetic acid, 5,6-Dimethoxy-1-indanone and methane sulphonic acid are added to the cooled reaction mixture as obtained above and heated to a temperature of about 80° C. to about 85° C. The reaction takes generally about 17 to 18 hrs for completion. The reaction mixture is cooled at about 10° C. to 15° C. and stirred for 30 min. The reaction mixture is filtered. The solid is washed with acetone and suck dried. The wet cake is added to acetone and slurry is made. The slurry is heated at a temperature of about 50° C. to about 55° C. for 30 min, cooled at ambient temperature and filtered. The solid is washed with acetone, suck dried and dried in oven at a temperature of about 45° C. to about 50° C. for 3 to 5 hours to give condensed bromide salt of formula (VI)

In the present invention, the condensed bromide salt is hydrogenated partially to give Di-ene intermediate of formula (VII). Partial hydrogenation can be achieved using mild reducing agents such as sodium borohydride. This di-ene intermediate is reduced to give donepezil. The advantage of doing two separate hydrogenation instead of single one given in prior art process is that the side reaction and generation of impurity is minimum and we get donepezil in high purity and also high yield.

To a cooled mixture of Condensed Bromide Salt, methanol and sodium carbonate at 3-5° C., a predissolved solution of Sodium borohydride and Sodium hydroxide in DM Water is added dropwise during 2 hrs at about 5° C. to about 15° C. The reaction mixture is stirred for 1 hour at the same temperature. A mixture of Acetone/Water (1:2) is slowly added and stirred for about 10 min. Methanol is distilled out below 45° C. till one third of the original volume remains. D M Water is added and heated at about 55° C. to about 60° C. for 30 min. The suspension is filtered hot, washed with D M water, suck dried and then dried in oven under vacuum at about 55° C. to about 60° C. for about 8 to 10 hrs to give crude Di-Ene. Di-ene crude is purified by crystallizing crude Di-ene from a mixture of ethyl acetate, acetone, D M Water and ammonia.

“Crystallization” as used herein includes processes in which a solution is rendered saturated or supersatured with respect to a dissolved component and the formation of crystals of this component is achieved. The initiation of crystal formation may be spontaneous, or it may require the addition of seed crystals. As used herein, crystallization or recrystallization also describes the situation in which a solid or liquid material is dissolved in a solvent to yield a solution which is then rendered saturated or supersatured so as to obtain crystals. Also, included in the term crystallization are the ancillary processes of washing the crystals with one or more solvents, drying the crystals, and harvesting the final product so obtained.

Crystallization can be achieved by the methods known in the art such as reducing the volume of the solution or cooling the solution or both.

Di-ene crude is added to a mixture of Ethyl acetate:Acetone (1:1) at ambient temperature. D M water and ammonia solution is added to it and the reaction mixture is heated to a temperature of about 60° C. to about 65° C. till clear solution is obtained. The mixture is cooled at about 0° C. to about 5° C. and stirred for 1 hour. The mixture is filtered, washed with chilled mixture of Acetone: Ethyl acetate (1:1), suck dried and dried under vacuum at a temperature of about 50° C. to about 55° C. for about 8 to 10 hrs to give pure Di-ene intermediate of formula (VII).

Di-ene is reduced to give donepezil free base by hydrogenation process. The hydrogenation is carried out using noble catalyst such as Palladium, platinum, ruthenium, rhodium or its chemical forms. The metal can be used supported on carbon in its 0 valent form or can be used in its chemically converted form such as PtO₂. In this step, slurry of PtO₂ in DM Water is added to a solution of Di-ene in methanol at ambient temperature. The reaction mixture is hydrogenated at pressure of about 4 to 5 kg of H₂ gas at a temperature of about 30° C. to about 35° C. for 2 hrs. The reaction mixture is monitored by HPLC. After completion of the reaction, the reaction mixture is filtered through hyflow bed. The bed is washed with methanol. The filtrate is evaporated to dryness under vacuum at below 40° C. to give oil. DM Water and Dichloromethane are added to the residue. The mixture is cooled at about 5° C. to about 10° C. and con. HCl is added. Dichloromethane is added to the reaction mixture and extracted. Both the layers are separated. Aq. layer is extracted with dichloromethane. The combined organic layer is washed with brine solution and distilled out. To the residue dichloromethane, DMF, DM Water is added. Acetone is added drop wise to it and stirred for about 8 to 10 hrs at ambient temperature. The mixture is cooled at about 10° C. to about 15° C. and stirred for 1 hour at the same temperature. The mixture is filtered, suck dried and dried under vacuum to a temperature of about 45° C. to about 50° C. for about 3 to 5 hrs to give the crude donepezil hydrochloride. Donepezil hydrochloride crude is added to ethanol and heated to a temperature of about 45° C. to about 50° C. DM Water is added to the mixture till clear solution. Activated carbon is added and stirred for 5 to 10 min at the same temperature. The reaction mixture is filtered hot through hyflow bed. The bed is washed with hot Ethanol. The filtrate is cooled to a temperature of about 5° C. to about 10° C. Diisopropylether is added slowly during 20 to 30 min to this cooled filtrate and stirred for about 1 to 2 hrs. The solution is optionally seeded with Donepezil Hydrochloride The mixture is filtered, washed with chilled diisopropylether and suck dried. The solid is dried to a temperature of about 45° C. to about 50° C. under vacuum for about 8 to 10 hrs to give donepezil hydrochloride. The donepezil hydrochloride obtained by the above process is Form I having XRD similar to that disclosed in U.S. Pat. No. 5,985,864.

The novel process for the preparation of donepezil hydrochloride of formula (I) can be described schematically as shown in Scheme-VI:

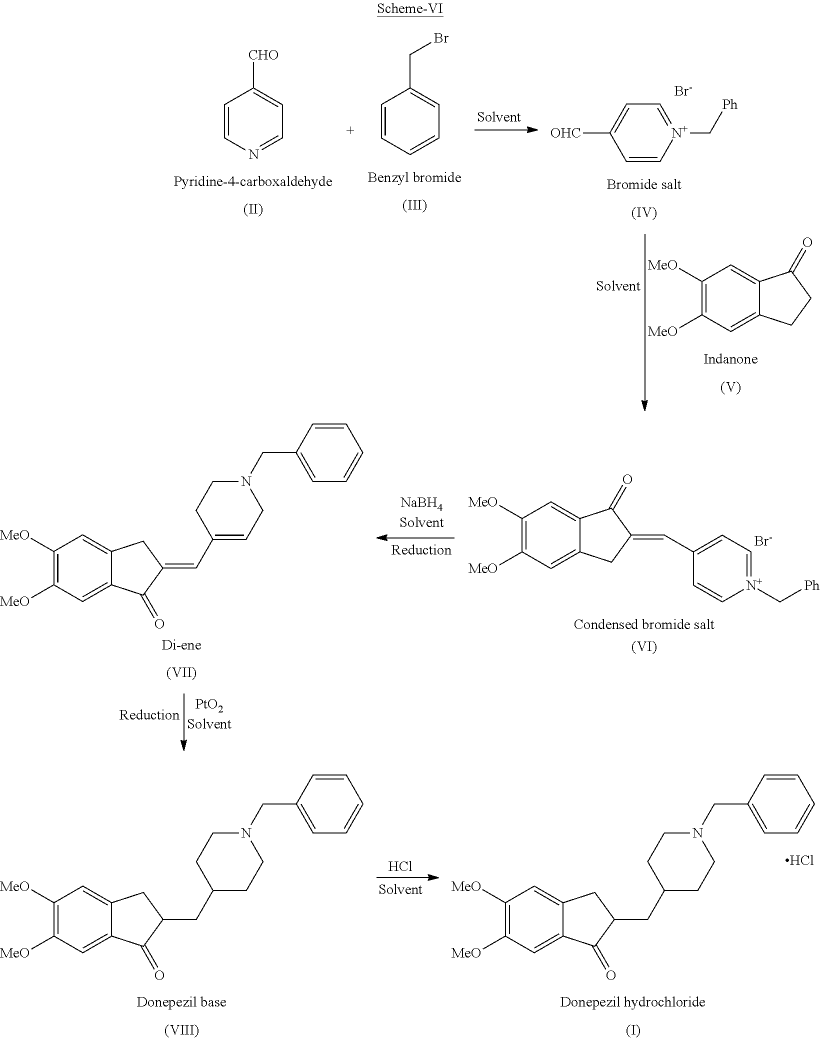

The following examples illustrate the invention further. It should be understood, however, that the invention is not confined to the specific limitations set forth in the individual examples but rather to the scope of the appended claims.

### Example-1

### Preparation of Condensed Bromide Salt

Benzyl Bromide (97.86 g) was added to a solution of Pyridine-4-carboxaldehyde (58.50 g) in N,N-dimethyl formamide (50 ml) at ambient temperature and then heated at 60-65° C. for 30 min. The reaction mixture was cooled at ambient temperature and acetic acid (500 ml), 5,6-Dimethoxy-1-indanone (100 g) and methane sulphonic acid (9.99 g) were added to the reaction mixture and heated to 80-85° C. for 17-18 hrs. The reaction mixture was cooled and filtered. The wet cake was washed with acetone. The wet cake was triturated with acetone at 50-55° C. for 30 min, cooled at ambient temperature and filtered. The solid was washed with acetone, suck dried and then dried in oven at 45-50° C. for 3-5 hours to give the condensed bromide salt (190-210 g).

### Example-2

### Preparation of Di-Ene

To a cooled mixture of Condensed Bromide Salt (Example 1) (100 g) in methanol (1200 ml) at 3-5° C., sodium carbonate (2.34 g), a predissolved solution of Sodium borohydride (19.27 g) and Sodium hydroxide (1.77 g) in DM Water (300 ml) was added dropwise during 2 hrs at 5-15° C. The reaction mixture was stirred at 5-15° C. for 1 h. A mixture of Acetone/Water [Acetone (50 ml) in D M water (100 ml)] was slowly added to the reaction mixture. Methanol was distilled out below 45° C. till residual volume 500 ml remains. D M Water (1000 ml) was added and heated to 55-60° C. for 30 min. The suspension was filtered hot, washed with D M water (100 ml), suck dried and then dried in oven under vacuum at 55-60° C. for 8-10 hours to give the Di-ene(75-85 g)

### Example-3

### Purification of Di-ene

Di-ene crude (Example 2) (100 g) was added to a mixture of Ethyl acetate (500 ml) and Acetone (500 ml) at ambient temperature. D M water (60 ml) and ammonia solution (20 ml) was added to it and heated at 60-65° C. till clear solution. The reaction mixture was cooled to 0-5° C. and stirred for 1 h. The reaction mixture was filtered, washed with chilled mixture of Acetone: Ethyl acetate (1:1) (50 ml), suck dried and dried under vacuum at 50-55° C. for 8-10 h to give the pure Di-ene (70-80 g).

### Example-4

### Preparation of Donepezil Hydrochloride Crude

To a mixture of Di-ene (Example 3) (100 g) in methanol (500 ml), a slurry of PtO₂ (2.5 g) in DM Water (50 ml) was added and hydrogenated at pressure 4-5 kg of H₂ gas for 2 hrs at 30-35° C. The progress of the reaction was monitored by HPLC. After completion of the reaction, the reaction mixture was filtered through hyflow bed. The bed was washed with methanol (2×50 ml). The filtrate was evaporated to dryness under vacuum at below 45° C. DM Water (300 ml) and Dichloromethane (50 ml) was added to the residue and cooled at 5-10° C. Con. HCl (30 ml) was added. Dichloromethane (250 ml) was added and extracted. Both the layers were separated. Aq. layer was extracted with dichloromethane (300 ml). The combined organic layer was washed with brine solution (2×200 ml) and then distilled out. To the residue dichloromethane (200 ml), DMF (200 ml), DM Water (50 ml) was added. Acetone (800 ml) was added dropwise to it and stirred for 8-10 hrs at 20-25° C. The mixture was cooled at 10-15° C. and stirred for 1 h. The mixture was filtered, suck dried and dried under vacuum at 45-50° C. for 3-5 hours to give crude donepezil hydrochloride (80-85 g).

### Example-4

### Donepezil Hydrochloride Form I

Donepezil hydrochloride crude (100 g) (Example 3) in Ethanol (800 ml) was heated at 45-50° C. DM Water (80 ml) was added to the reaction mixture and heated at 45-50° C. till clear solution was obtained. Activated carbon (2 g) was added and stirred for 5-10 min at the same temperature. The reaction mixture was filtered hot through hyflow bed. The bed was washed with hot ethanol (2×100 ml). The filtrate was cooled at 5-10° C. To this filtrate, diisopropylether (1200 ml) was added slowly during 20-30 min at 5-10° C. and then stirred for 1-2 h at 5-10° C. The reaction mixture was filtered, washed with chilled diisopropylether (50 ml) and suck dried. The solid was dried at 40-45° C. under vacuum for 8-10 hrs to give donepezil hydrochloride Form I (85-95 g).

What is claimed is:

1. A process for preparation of Donepezil hydrochloride of formula (I) 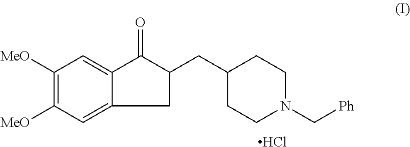
comprising a step of condensing bromide salt of formula (IV) with indanone of formula (V) 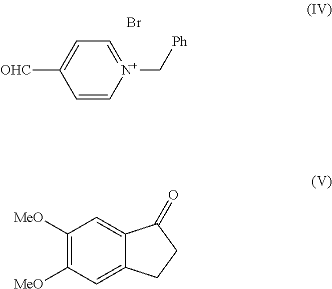
to obtain condensed bromide salt of formula (VI).

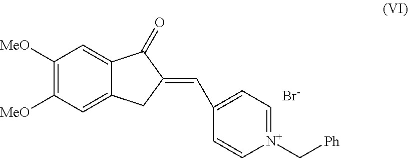

2. A process for preparation of Donepezil hydrochloride of formula (I) 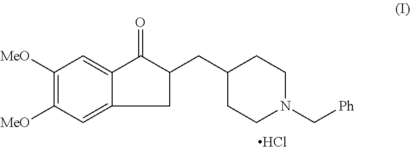
comprising steps of:
(a) condensing pyridine-4-carboxaldehyde of formula (II) with benzyl bromide of formula (III) to obtain bromide salt of formula (IV)
(b) condensing bromide salt of formula (IV) in situ with indanone of formula (v) to obtain condensed bromide salt of formula (VI)

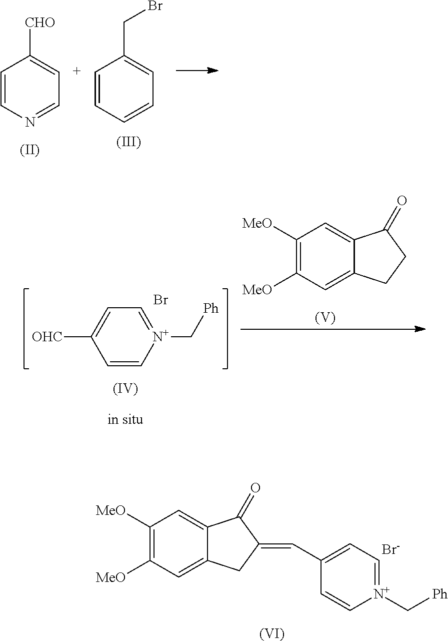

3. A process for preparation of Donepezil hydrochloride of formula (I) 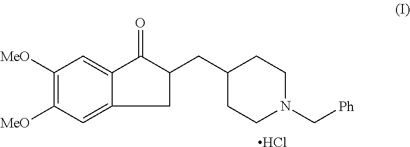
comprising a step of reducing condensed bromide salt of formula (VI) to obtain Di-Ene intermediate of formula (VII).

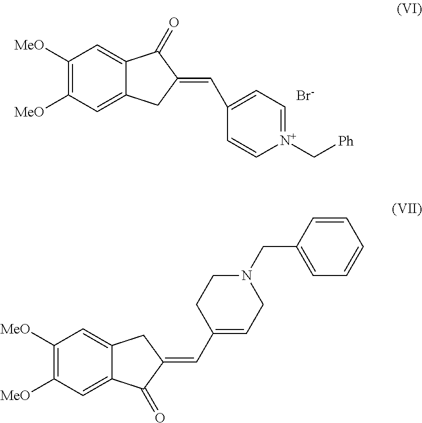

4. A process as claimed in claim 3, wherein reducing agent is sodium borohydride.

5. A process for preparation of Donepezil hydrochloride of formula (I) 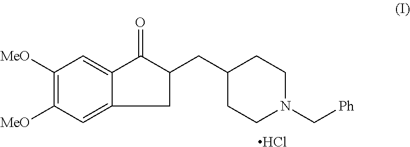
comprising a step of reducing Di-Ene intermediate of formula (VII) to obtain donepezil free base of formula (VIII).

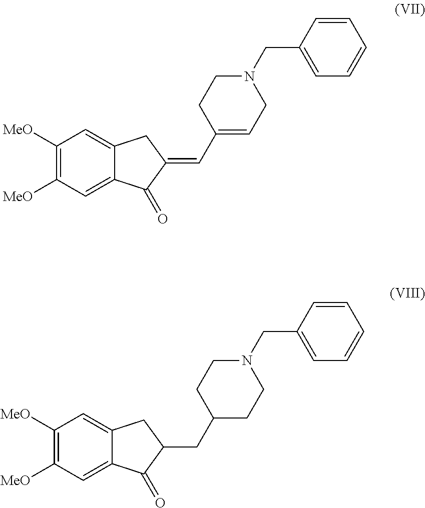

6. A process as claimed in claim 5, wherein reduction is carried out by hydrogenation using noble metal catalyst such as Palladium, platinum, ruthenium, rhodium or its chemical forms.

7. A process as claimed in claim 5, wherein reduction is carried out using hydrogenation over platinum oxide catalyst.

8. A novel Di-Ene intermediate of formula (VII)

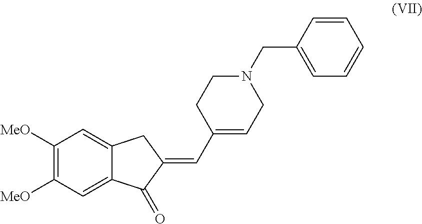

9. Use of novel intermediate of formula (VII) for the preparation of Donepezil hydrochloride of formula (I)

10. A process for preparation of Donepezil hydrochloride of formula (I) 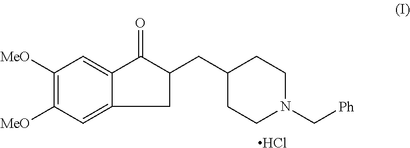
comprising purification of Di-ene intermediate of formula (VII) 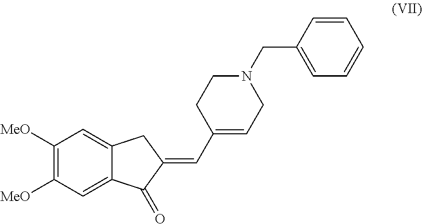
comprising crystallizing crude Di-ene from a mixture of ethyl acetate, acetone, D M Water and ammonia.
